(19) United States
(12) Patent Application Publication
(10) Pub. No.: US 2006/0263512 A1
(43) Pub. Date: Nov. 23, 2006

(54) MULTI-LAYER COATING SYSTEM AND METHOD

(76) Inventor: David A. Glocker, West Henrietta, NY (US)

Correspondence Address: SIMPSON & SIMPSON, PLLC, 5555 MAIN STREET, WILLIAMSVILLE, NY, 14221-5406, US

(21) Appl. No.: 11/419,038

(22) Filed: May 18, 2006

Related U.S. Application Data
(60) Provisional application No. 60/682,734, filed on May 19, 2005.

(51) Int. Cl. A61L 33/00

(52) U.S. Cl. 427/2.25

(57) ABSTRACT

A system and method for coating implantable medical devices so that they do not interfere with MR imaging are described. Using any of the coating processes well known to those skilled in the art, e.g., physical vapor deposition such as evaporation, sputtering, or cathode arc, or chemical vapor deposition, spraying, plasma polymerization, plasma enhanced chemical vapor deposition and the like, multiple sources, including at least one source of an electrically insulating material and at least one source of an electrically conducting material, are oriented and shielded so as to coat separate sections of the implantable medical device. The object being coated is then rotated so that overlapping spiral coatings of the materials from the different coating sources are produced on the object.

### CROSS-REFERENCE TO RELATED APPLICATION

This utility patent application claims the benefit of U.S. Provisional Patent Application No. 60/682,734, filed on May 19, 2005, the entire disclosure of which is incorporated herein for any and all purposes.

### BACKGROUND OF THE INVENTION

Magnetic Resonance Imaging (MRI) is extensively used to non-invasively diagnose patient medical problems. The patient is positioned in the aperture of a large annular magnet that produces a strong and static magnetic field. The spins of the atomic nuclei of the patient's tissue molecules are aligned by the strong static magnetic field. Radio frequency pulses are then applied in a plane perpendicular to the static magnetic field lines so as to cause some of the hydrogen nuclei to change alignment. The frequency of the radio wave pulses used is governed by the Larmor Equation. Magnetic field gradients are then applied in 3 orthogonal directions to allow encoding of the position of the atoms. At the end of the radio frequency pulse the nuclei return to their original configuration and, as they do so, they release radio frequency energy, which can be picked up by coils surrounding the patient. These signals are recorded and the resulting data are processed by a computer to generate an image of the tissue. Thus, the examined tissue can be seen with its quite detailed anatomical features. In clinical practice, MRI is used to distinguish pathologic tissue such as a brain tumor from normal tissue.

The technique most frequently relies on the relaxation properties of magnetically excited hydrogen nuclei in water. The sample is briefly exposed to a burst of radiofrequency energy, which in the presence of a magnetic field puts the nuclei in an elevated energy state. As the molecules undergo their normal, microscopic tumbling, they shed this energy to their surroundings in a process referred to as “relaxation.” Molecules free to tumble more rapidly relax more rapidly.

T1-weighted MRI scans rely on relaxation in the longitudinal plane, and T2 weighted MRI scans rely on relaxation in the transverse plane. Differences in relaxation rates are the basis of MRI images—for example, the water molecules in blood are free to tumble more rapidly, and hence, relax at a different rate than water molecules in other tissues. Different scan sequences allow different tissue types and pathologies to be highlighted.

MRI allows manipulation of spins in many different ways, each yielding a specific type of image contrast and information. With the same machine a variety of scans can be made and a typical MRI examination consists of several such scans.

One of the advantages of a MRI scan is that, according to current medical knowledge, it is harmless to the patient. It only utilizes strong magnetic fields and non-ionizing radiation in the radio frequency range. Compare this to CT scans and traditional X-rays which involve doses of ionizing radiation. It must be noted, however, that the presence of a ferromagnetic foreign body (say, shell fragments) in the patient, or a metallic implant (like surgical prostheses, or pacemakers) can present a (relative or absolute) contraindication towards MRI scanning: interaction of the magnetic and radiofrequency fields with such an object can lead to mechanical or thermal injury, or failure of an implanted device.

Even if implanted medical devices pose no danger to the patient, they may prevent a useful MR image from being obtained, due to their perturbation of the static, gradient and/or radio frequency pulsed magnetic fields and/or the response signal from the imaged tissue. Examples of problems encountered when attempting to use MRI to image tissue adjacent to implanted medical devices are discussed in U.S. Pat. No. 6,712,844, the entire disclosure of which is hereby incorporated by reference into this specification. U.S. Pat. No. 6,712,844 states “While researching heart problems, it was found that all the currently used metal stents distorted the magnetic resonance images. As a result, it was impossible to study the blood flow in the stents which were placed inside blood vessels and the area directly around the stents for determining tissue response to different stents in the heart region.” U.S. Pat. No. 6,712,844 goes on to state “It was found that metal of the stents distorted the magnetic resonance images of blood vessels. The quality of the medical diagnosis depends on the quality of the MRI images. A proper shift of the spins of protons in different tissues produces high quality MRI images. The spin of the protons is influenced by radio frequency (RF) pulses, which are blocked by eddy currents circulating at the surface of the wall of the stent. The RF pulses are not capable of penetrating the conventional metal stents. Similarly, if the eddy currents reduce the amplitudes of the radio frequency pulses, the RF pulses will lose their ability to influence the spins of the protons. The signal-to-noise ratio becomes too low to produce any quality images inside the stent. The high level of noise to signal is proportional to the eddy current magnitude, which depends on the amount and conductivity of the stent in which the eddy currents are induced and the magnitude of the pulsed field.”

The currents induced in implanted metallic stents, and other devices, by the incident radio frequency radiation in the MRI field create, according to Lenz's law, magnetic fields that oppose the change of the magnetic fields of the incident radiation, thereby distorting and/or reducing the contrast of the resulting image.

Examples of attempts to improve the images in and around stents in MRI by incorporating resonance circuits with the stents are found, i.e., in U.S. Pat. No. 6,280,385 (“Stent and MR Imaging Process for the Imaging and the Determination of the Position of a Stent”) and U.S. Pat. No. 6,767,360 (“Vascular Stent with Composite Structure for Magnetic Resonance Imaging Capabilities”). The entire disclosure of each of these United States patents is hereby incorporated by reference into this specification.

U.S. Pat. No. 6,280,385 states in column 3, lines 29-44: “These and other objects are achieved by the present invention, which comprises a stent which is to be introduced into the examination object. The stent is provided with an integrated resonance circuit, which induces a changed response signal in a locally defined area in or around the stent that is imaged by spatial resolution. The resonance frequency is essentially equal to the resonance frequency of the applied high-frequency radiation of the magnetic resonance imaging system. Since that area is immediately adjacent to the stent (either inside or outside thereof), the position of the stent is clearly recognizable in the correspondingly enhanced area in the magnetic resonance image. Because a changed signal response of the examined object is induced by itself, only those artifacts can appear that are produced by the material of the stent itself.” claim 1 in column 12 of U.S. Pat. No. 6,280,385 claims: “1. A magnetic resonance imaging process for the imaging and determination of the position of a stent introduced into an examination object, the process comprising the steps of: placing the examination object in a magnetic field, the examination object having a stent with at least one passive resonance circuit disposed therein; applying high-frequency radiation of a specific resonance frequency to the examination object such that transitions between spin energy levels of atomic nuclei of the examination object are excited; and detecting magnetic resonance signals thus produced as signal responses by a receiving coil and imaging the detected signal responses; wherein, in a locally defined area proximate the stent, a changed signal response is produced by the at least one passive resonance circuit of the stent, the passive resonance circuit comprising an inductor and a capacitor forming a closed-loop coil arrangement such that the resonance frequency of the passive resonance circuit is essentially equal to the resonance frequency of the applied high-frequency radiation and such that the area is imaged using the changed signal response.”

U.S. Pat. No. 6,767,360 states in column 2, lines 29-39: “Imaging procedures using MRI without need for contrast dye are emerging in the practice. But a current considerable factor weighing against the use of magnetic resonance imaging techniques to visualize implanted stents composed of ferromagnetic or electrically conductive materials is the inhibiting effect of such materials. These materials cause sufficient distortion of the magnetic resonance field to preclude imaging the interior of the stent. This effect is attributable to their Faraday physical properties in relation to the electromagnetic energy applied during the MRI process.” U.S. Pat. No. 6,767,360 further states in column 2, lines 50-64: “In German application 197 46 735.0, which was filed as international patent application PCT/DE98/03045, published Apr. 22, 1999 as WO 99/19738, Melzer et al (Melzer, or the 99/19738 publication) disclose an MRI process for representing and determining the position of a stent, in which the stent has at least one passive oscillating circuit with an inductor and a capacitor. According to Melzer, the resonance frequency of this circuit substantially corresponds to the resonance frequency of the injected high-frequency radiation from the magnetic resonance system, so that in a locally limited area situated inside or around the stent, a modified signal answer is generated which is represented with spatial resolution. However, the Melzer solution lacks a suitable integration of an LC circuit within the stent.”

Claims 1 and 2 in column 9 of U.S. Pat. No. 6,767,360 claim: “1. A stent adapted to be implanted in a duct of a human body to maintain an open lumen at the implant site, and to allow viewing body properties outside and within the implanted stent by magnetic resonance imaging (MRI) energy applied external to the body, said stent comprising a metal scaffold, and an electrical circuit resonant at the resonance frequency of said MRI energy integral with said scaffold. 2. A stent adapted to be implanted in a duct of a human body to maintain an open lumen at the implant site, said stent comprising a tubular scaffold of low ferromagnetic metal, and an inductance-capacitance (LC) circuit integral with said scaffold, said LC circuit being geometrically structured in combination with said scaffold to be resonant at the resonance frequency of magnetic resonance imaging (MRI) energy to be applied to said body to enable MRI viewing of body tissue and fluid within the lumen of the stent when implanted and subjected to said MRI energy.”

WO 02/085216 A1, which is incorporated herein by reference, recognizes the need for enhanced imaging in the vicinity of a biopsy needle or other interventional medical device. However, the inventors address that need by describing an antenna that is inserted into the examination object to receive signals from the excited protons. The antenna is connected through a coaxial cable to circuitry external to the examination object. U.S. Pat. No. 5,447,156 describes an “RF transmitter means attached to (an) RF coil within the MR-active invasive device for transmitting RF energy into said subject of a selected duration, amplitude and frequency to cause nutation of a second selected ensemble of spins.” Both of these inventions require signals to be coupled either into or out of the examination device to improve the image quality.

U.S. patent application Ser. No. 11/132,469 titled “Device Compatible with Magnetic Resonance Imaging” describes “a plurality of coated layers . . . disposed on an implanted device. The material and electrical parameters of the coated layers are chosen and the geometry of the coated layers is arranged so that incident electromagnetic radiation induces currents in the coated layers that have a predetermined phase and amplitude relationship with the current induced in the implanted device.” The Application further describes the use of a two-layer structure coated in a spiral pattern to achieve this.

In addition to achieving the proper electrical characteristics, the coatings must be able to withstand the significant stresses that biomedical devices must undergo in use. For example, stents are often made of an alloy of nickel and titanium, known as Nitinol. The unusual super-elastic and shape memory properties of Nitinol are well-known and are the result of the fact that Nitinol undergoes a transformation from a martensitic phase to an austenitic phase as a consequence of temperature changes or stress. In fact, Nitinol must often undergo strains of up to approximately 8% when use in medical devices. Therefore, in order to perform any coating applied to such devices must also be able to undergo similar strains, which presents a significant challenge.

### SUMMARY OF THE INVENTION

A system for coating a medical device for use within a subject so that the device is capable of being imaged using magnetic resonance, the system comprises a medical device; a source of an electrically conducting material positioned to coat at least a portion of the medical device; a source of an electrically insulating material positioned to coat at least a portion of the medical device; at least one shield isolating the electrically conducting material from the electrically insulating material; and a device for rotating the medical device relative to the conducting material and the insulating material.

A method for coating a medical device for use within a subject so that the device is capable of being imaged using magnetic resonance, the method comprises positioning a source of an electrically conducting material to coat at least a portion of a medical device; positioning a source of an electrically insulating material to coat at least a portion of the medical device; shielding the electrically conducting material from the electrically insulating material; and rotating the medical device relative to the electrically conducting material and the electrically insulating material.

### BRIEF DESCRIPTION OF THE DRAWINGS

In the detailed description of the preferred embodiments of the invention presented below, reference is made to the accompanying drawings, in some of which the relative relationships of the various components are illustrated, it being understood that orientation of the apparatus may be modified. For clarity of understanding of the drawings, relative proportions depicted or indicated of the various elements of which disclosed members are comprised may not be representative of the actual proportions, and some of the dimensions may be selectively exaggerated.

FIGS. 1A-C are schematic diagrams of stents.

FIG. 2 a stent coated using one preferred embodiment of the present invention.

FIG. 2A another stent coated using another preferred embodiment with a smaller overlap angle than shown in FIG. 2.

FIG. 3 is a schematic illustration of a coating apparatus according to the present invention for producing the coated object in FIG. 2 or FIG. 2A.

FIG. 3A is a schematic illustration of a coating apparatus according to the present invention for producing several coated objects at once.

FIG. 3B is a schematic illustration of a cylindrically symmetric apparatus according to the present invention for producing several coated objects at once.

FIG. 4 is a simplified illustration of a model used to predict the performance of coatings made according to the present invention.

FIG. 5 illustrates the geometry used to calculate the performance of a ring coating made according to the present invention.

FIG. 6 illustrates the results of resonance measurements made on a coating deposited using one preferred embodiment of the present invention.

### DETAILED DESCRIPTION OF THE INVENTION

A stent is an expandable tubular mesh structure that is inserted into a lumen structure of the body to keep it open. Stents are used in diverse structures in the body such as the esophagus, trachea, blood vessels, and the like. Prior to use, a stent is collapsed to a small diameter. When brought into place it is expanded either by using an inflatable balloon or is self-expending due to the elasticity of the material. Once expanded the stent is held in place by its own material strength. Stents are usually inserted by endoscopy or other procedures less invasive than a surgical operation. Stents are typically metallic, for example, stainless steel, alloys of nickel and titanium, or the like and are therefore electrically conducting.

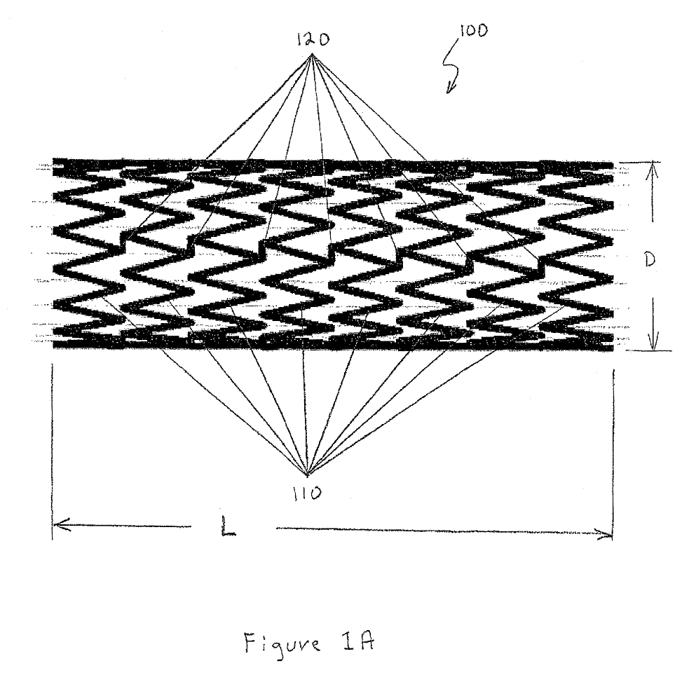

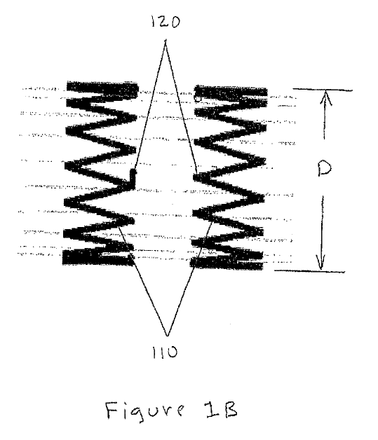

FIG. 1A is a schematic illustration of one embodiment of a stent to which the invention may be applied. FIG. 1A is a side elevational view of a tubular stent 100 having a length L and a diameter D. Stent 100 is comprised of a plurality of electrically conducting, sawtooth shaped circumferential loops 110, each loop 110 connected to the next loop 110 at a plurality of points 120 around the circumference of each loop 110. In stent 100 of FIG. 1A each loop 110 is connected to the next loop 110 at four points 120 around the circumference, but only one of the four connection points can be seen in the side elevational view of FIG. 1A. FIG. 1B is a schematic illustration of two of the circumferential loops 110 separated from each other. Other embodiments of stents to which the invention may be applied may have sawtoothed shaped circumferential loops attached to each other at more points around the circumference. FIG. 1C, for example, shows a schematic side elevational view of a stent 150 in which the sawtooth shaped circumferential loops are attached to each other at every sawtooth apex.

It should be apparent from the above description of the stents depicted in FIGS. 1A and 1C that one can trace many different closed loop conducting paths in either of those stents. For example, a circular closed loop-conducting path may be traced around each sawtoothed shaped circumferential loop 110. It should also be apparent that one could trace longitudinal conducting paths in either stent 100 in FIG. 1A or stent 150 in FIG. 1C by moving from one circumferential loop 110 to the next circumferential loop 110 through the connection points 120 in the same longitudinal row. In stent 100 in FIG. 1A there are four such longitudinal conducting paths along the four rows of connection points spaced at 90° intervals around the circumference of stent 100. In stent 150 in FIG. 1C there could be many such longitudinal conducting paths. Furthermore, in stent 100 in FIG. 1A or stent 150 in FIG. 1C, one may trace helical conducting paths by moving from one circumferential loop 10 to the next circumferential loop 110 at connecting points in successively different longitudinal rows.

While stents as illustrated in FIGS. 1A and 1C are common, the invention is not limited to stents comprising connected sawtooth shaped circumferential loops. The invention may be applied to any tubular stent in which closed loop conducting loops can be traced. Other implantable medical devices such as pacemakers and the like may also be coated by the method of the invention.

When either of stents 100 or 150 is implanted in a subject and placed in a MRI field, the varying magnetic field of the MRI gradient and radio frequency imaging radiation will induce currents in the conducting tubular mesh structure of stent 100 or 150. As described above, many closed loop conducting paths exist in stent 100 or 150 in which such induced currents could flow. Such induced currents produce, via Lenz's law, varying magnetic fields that oppose the varying magnetic fields of the incident RF radiation, thereby distorting and/or reducing the contrast of the resulting magnetic resonance image. For the sake of simplicity, in the following detailed description of the invention, the embodiments of the invention will be described in terms of coatings disposed on a single conducting circular ring. The single conducting circular ring will serve as a surrogate for any of the closed loop conducting paths in stents 100 or 150 as described above.

While the embodiments of the invention will be described in terms of coatings disposed on a single conducting circular ring, it will be obvious to those of ordinary skill in the art that such embodiments can be extended to the structure of stent 100 or 150 in FIGS. 1A and 1C respectively. Additionally, it should be obvious to those of ordinary skill in the art that embodiments described in terms of coatings disposed on a single conducting circular ring may also be extended to any situation in which electromagnetic radiation is incident on any device comprised of a conducting substrate with one or more holes therein. The perimeter of each such hole is the analog of a single conducting circular ring.

Other preferred embodiments are coatings on devices other than stents, such as catheters, guidewires and the like, to provide markers that enhance their visibility in an MRI image and to improve the image quality in a specific location. A medical device with a coating that resonates at the applied RF imaging radiation frequency, typically approximately 64 or 128 MHz, would cause the oscillating magnetic field in the region of the coating to have a greater strength than the oscillating field elsewhere. This increased field strength will cause the tissue in the vicinity of the coating to have a greater number of excited spins, resulting in a greater image strength in that location. That will make the coating easy to see and serve as a marker for its location, which would aid in locating the device within the examination object.

In addition, the increased oscillating field strength in the vicinity of the inventive coating will cause the net magnetization of the spins in that region to have a greater transverse component (become more perpendicular to the applied static field) than in other regions. Such an increase in transverse magnetization, or increased flip angle as it is sometimes called, is known to improve the signal to noise ratio (SNR) in MRI. Therefore, by manipulating a device with the inventive coating into a position within the examination object where greater detail is desired, the coating can be used to improve the image quality in areas that are of particular interest or importance. The coating can be applied to an object with a solid core, so that the region external to the coating is visible, or it can be applied to a hollow tube, in which case the region within the tube may also be imaged.

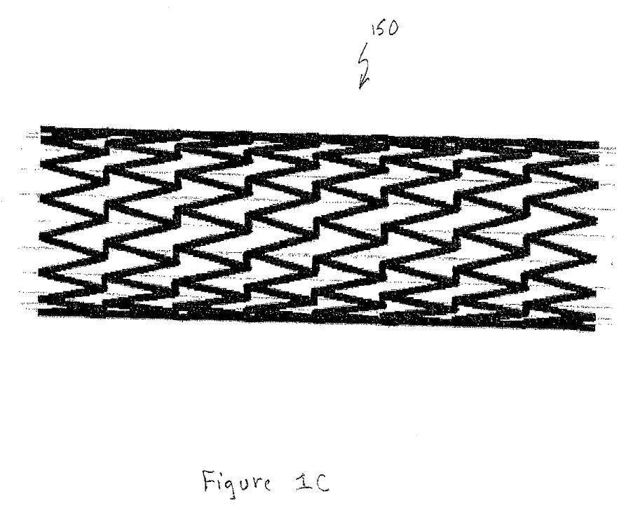

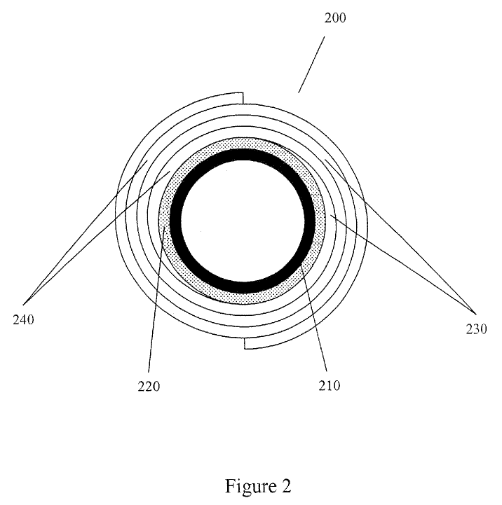

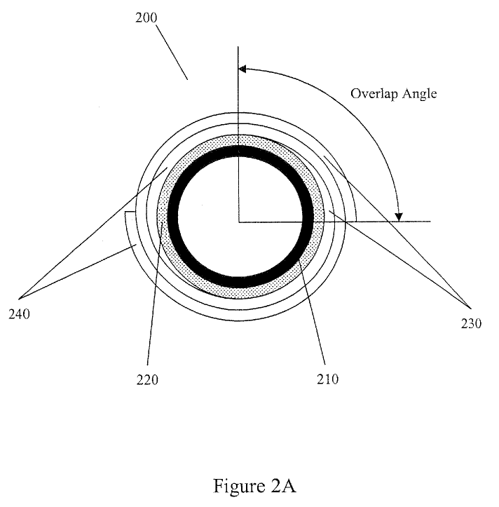

One embodiment of a medical device made according to the present invention is the coated ring assembly depicted schematically in FIG. 2. Referring to FIG. 2, there is shown a cross-sectional view of a coated ring assembly 200 comprising a conducting ring 210 coated with a plurality of coated layers 220, 230, and 240. Conducting ring 210 is first completely coated with a first electrically insulating layer 220. First insulating layer 220 is then coated with a first electrically conducting layer 230 in a spiral fashion. A second insulating layer 240 is also coated in a spiral fashion over conducting ring 210 so that it is interleaved with conducting layer 230.

When coated ring assembly 200 is placed in a MRI field, the RF imaging radiation of the MRI field will induce currents in conducting ring 210. As discussed above, such induced currents in ring 210 produce induced RF magnetic fields that oppose the incident MRI RF magnetic fields that produced the induced currents and, as a result, distort or even obliterate the MR images. However, in response to the RF imaging radiation currents will also be induced in conducting layer 230 and displacement currents will be produced across the insulating layer 240. The result is that interleaved spiral layers 230 and 240 respond electrically as an RLC circuit. As described in U.S. patent application Ser. No. 11/132,469, incorporated herein by reference for any and all purposes, it is believed that layers 220, 230, and 240 may be modeled as an equivalent, inductively coupled, RLC circuit driven by the incident RF imaging radiation of the MRI field. The equivalent values of R, L, and C will determine the phase and amplitude relationship between the currents induced in layers 230 and 240 and the current induced in the ring 210.

As further described in U.S. patent application Ser. No. 11/132,469, in one embodiment, it is desired that the current induced in the combination of layers 230 and 240 be nearly in phase with, and nearly the same amplitude as, the current induced in the ring 210. In another embodiment it is desired that the current induced in the combination of layers 230 and 240 be out of phase and differ in amplitude, by predetermined amounts, with the current induced in the ring 210. The phase and amplitude relationship between the currents induced in the combination of layers 230 and 240 and the current induced in the ring 210 depends upon the relationship of the frequency of the RF imaging radiation to the resonant frequency of the equivalent RLC circuit of the coated ring assembly 200.

In some applications, such as a marker or to enhance the SNR of the local image, it may be useful to deposit the inventive spiral coating on an electrically insulating substrate such as a ceramic or a polymer. For example, catheters are often made of polymers. In such cases the electrical response of the coated object will be due entirely to the inductance, capacitance and resistance of the coating. If the substrate 210 in FIG. 2 is electrically insulating, there is no need to coat an insulating layer 220 prior to layers 230 and 240.

FIG. 2A shows a spiral coating according to the present invention in which the overlap angle (the angle between the beginning and end of the conducting layer 230) is approximately 90 degrees. The overlap angle will always be greater than approximately zero degrees because there must be some overlap of the ends of the conducting layer separated by the insulating layer in order to produce a capacitive reactance for the coating. Selection of the parameters of the insulating and conducting materials, such as coated thickness, dielectric constant, and conductivity, in addition to the overlap angle of the coating, allows considerable flexibility in producing a coated assembly having specific equivalent RLC circuit properties.

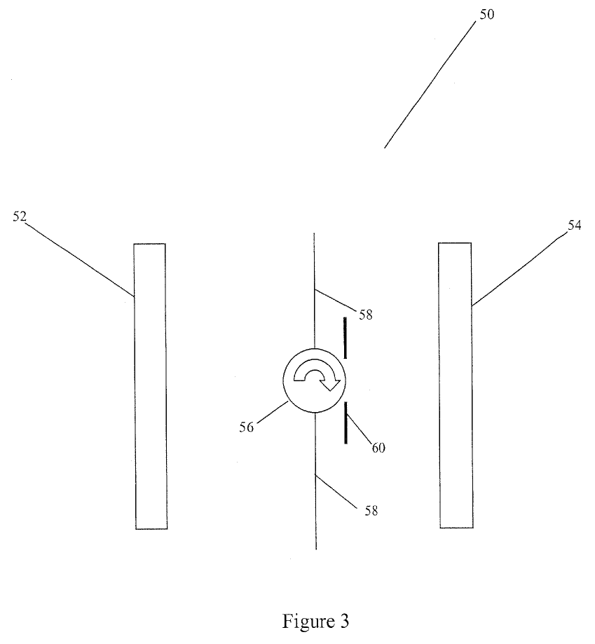

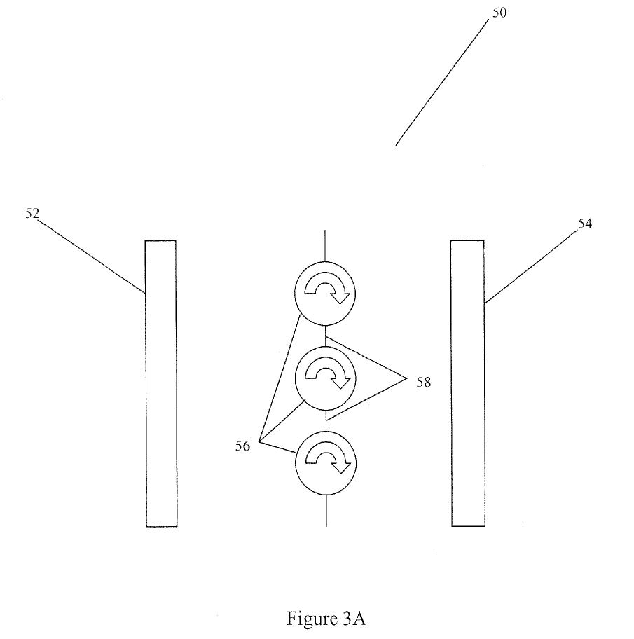

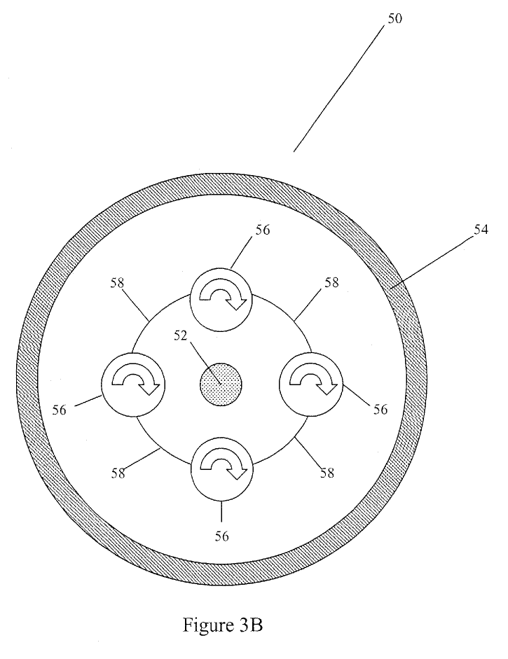

The two-material spiral coatings in FIG. 2 or FIG. 2A may be coated by any one of the coating techniques mentioned above by the process depicted in FIG. 3. Referring to FIG. 3, in order to produce a continuous spiral coating of two different materials, in the fashion as depicted in FIG. 2 or FIG. 2A, the coating setup 50 is used. Two different materials, material A and material B, are simultaneously deposited from sources 52 and 54 respectively. Sources 52 or 54 may be physical vapor deposition sources, such as evaporators, sputtering targets, or cathodic arc targets. Alternatively, sources 52 or 54 may be chemical vapor deposition sources, spray sources, thermal polymerization sources, or the like. Furthermore, the two sources may be of different types. For example, source 52 may be a physical vapor deposition source while source 54 may be a plasma polymerization source. The source of electrically conducting material may comprise Au, Ag, Cu, Ti, Pt, Pd and/or Nitinol, for example. The source of electrically insulating material may comprise a monomer that is cured using an electron beam, ultraviolet light, or the like, for example. Alternatively, the source of electrically insulating material may comprise an evaporated or vacuum deposited polymer, such as parylene, for example. The source of electrically insulating material may also comprise a metal oxide, such as Al₂O₃, TiO₂ or Ta₂O₅ or a nitride such as AlN. Further materials that can comprise the source of electrically insulating material include polymers, such as an acrylate that can be cured with electrons, ultraviolet light or other means, and plasma polmerizable materials, such as hexamethyldisiloxane, tetraethoxysilane, hexamethylcyclotrisiloxane, polytetrafluroethylene, and the like.

The substrate 56 to be coated is located between sources 52 and 54 and is rotated continuously during the coating process. Shields 58 are located so as to prevent the material from source 52 from mixing with the material from source 54. Masks 60 may be used to restrict the coatings from either or both sources 52 and 54 to certain regions of substrate 56 in a method well known in the art. Those regions may be different for sources 52 and 54.

FIG. 3A shows another embodiment in which multiple substrates are being coated simultaneously by positioning them side by side in front of two material sources. FIG. 3B shows still another embodiment in which multiple substrates are being coated in a circular fashion. In FIG. 3B, a first material comes from a source 52 that radiates outward, such as a post magnetron sputtering cathode or a long evaporation filament, both of which are well known to those skilled in the art. And a second material in FIG. 3B comes from a source 54 that radiates inward, such as an inverted cylindrical magnetron sputtering source, an ultraviolet light source to cure a monomer that is being fed into the outer chamber, or other such means well known to those skilled in the art. In FIG. 3B, either the inner or outer source could deposit the conducting material. In both FIGS. 3A and 3B, barriers 58 serve to isolate the material sources and multiple substrates 56 rotate about their axes. Masks, not shown in 3A and 3B, may also be used.

Without wishing to be bound by any particular theory, applicant has analyzed the two-material spiral coating represented in FIG. 2 or FIG. 2A as follows. Consider the case that FIG. 2A is a cross-section of a cylindrical conducting tube 210, whose length is much greater than its diameter, with associated coatings 220, 230 and 240. We will model the coating as simply a sheet of current being carried in conductor 230 with ends that overlap and are separated by an insulator.

Ampere's Law states that the line integral of the magnetic field around any closed path is equal to a constant times the current the path encloses, or [symbol]
\vec{B}·\vec{ds}=μ₀I
If we have a long cylindrical conductor carrying an azimuthal sheet of current I, neglecting end effects the magnetic field B is constant inside and zero outside. If the length of the cylinder is d, by integrating around a closed path that encloses the current sheet Ampere's law becomes

$$B = {μ}_{o} ( \frac{I}{d} )$$

The flux through the cylinder φ is BA, where A is the area of the cylinder. Therefore,

$$ϕ = \frac{{μ}_{o} IA}{d} .$$

The self-inductance of the sheet L is φ/I, so

$$L = \frac{{μ}_{o} A}{d} .$$

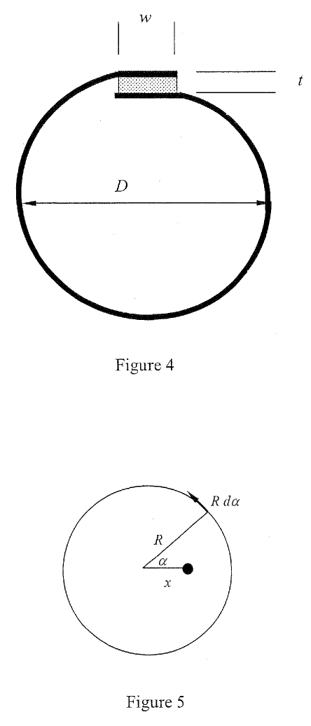

Assume that the sheet is cut along its length and allowed to overlap by a width w and that the overlapped ends are separated by a dielectric material of thickness t and relative dielectric constant εᵣ, as shown in FIG. 4. (The overlap width w is simply the overlap angle measured in radians, which was defined earlier, times the radius of the conducting sheet. FIG. 4 is a simplified version of the portion of the spiral coating in FIG. 2A that shows only the conducting layer and the overlap.) In that case the capacitance of the overlap is given by

$$C = {ɛ}_{o} {ɛ}_{r} \frac{wd}{t} .$$

Therefore, the LC constant of the overlapped sheet is approximately

$$LC = {μ}_{o} {ɛ}_{o} {ɛ}_{r} \frac{wA}{t} = {μ}_{o} {ɛ}_{o} {ɛ}_{r} \frac{π {D}^{2} w}{4 t} ,$$

where D is the diameter of the sheet. Tn order for this coating to resonate at a frequency f,

$$2 π f = \frac{1}{\sqrt{LC}} , or \\ \frac{{D}^{2} w}{t} = \frac{1}{{μ}_{o} {ɛ}_{o} {ɛ}_{r} {π}^{3} {f}^{2}} . \qquad Equation 1$$

For example, for a resonant frequency of 64 MHz and a relative dielectric constant εᵣ of 3, this becomes

$$\frac{{D}^{2} w}{t} = 0.24 {m}^{2} .$$

The plot below shows the overlap width in mm for resonance at 64 MHz as a function of the dielectric thickness given a relative dielectric constant of 3. The results for three sheet diameters, 0.5, 1.0 and 2.0 cm, are shown. [image]
As the length of the cylinder becomes less, the assumptions leading to these results break down. If the length becomes much less than the diameter, the sheet becomes a ring of current. In this case, the Biot-Savart law can be used to estimate the flux through the loop for a given current I.

For a closed loop carrying a current I, the contribution to the magnetic field at any point due to an infinitesimal segment of the loop is given by

$$d \vec{B} = ( \frac{{μ}_{o} I}{4 π {r}^{2}} ) d \vec{l} \times \hat{r} ,$$

where r is distance from the segment to the point where the field is measured, d\vec{l} is the length of the segment and \hat{r} is a unit vector pointing from the segment to the position where the field is measured. Integrating this in general is very complex. However, in the plane of a flat circular loop the expression simplifies. In that case, dB is perpendicular to the plane of the loop everywhere with a magnitude given by

$$dB = \frac{{μ}_{o} IR}{4 π} [ \frac{\sqrt{1 - \frac{{x}^{2} {sin}^{2} α}{( {R}^{2} + {x}^{2} - 2 Rx cos α )}}}{{R}^{2} + {x}^{2} - 2 Rx cos α} ] d α .$$

In this expression x is the distance from the center of the loop to where the field is measured, R is the radius of the loop, and R dα is the vector d\vec{l}, as shown in FIG. 5. Integrating gives us B(x). The flux through the loop can then be calculated by using

$$ϕ = {\int }_{0}^{R} B ( x ) 2 π x ⅆ x .$$

The self-inductance, which is the flux per unit current, is therefore given by

$${\int }_{0}^{R} \frac{{μ}_{o} Rx}{2} {\int }_{0}^{2 π} \frac{\sqrt{1 - \frac{{x}^{2} {sin}^{2} α}{( {R}^{2} + {x}^{2} - 2 Rx cos α )}}}{( {R}^{2} + {x}^{2} - 2 Rx cos α )} ⅆ α ⅆ x$$

The plot below shows the self inductance of a ring as a function of its diameter, calculated from the expression above. [image]
The relationship is L=KD, where the constant K is 5×10⁻⁶ H/m. In keeping with the previous calculations, we will let d be the width of the ring. Therefore, if the overlap width is w before, the overlap area will be wd. For resonance at 64 MHz,
LC=6.2×10⁻¹⁸ s²
Combining this with the expression for the capacitance,

$$A {ɛ}_{o} {ɛ}_{r} \frac{Dwd}{t} = 6 … 2 \times {10}^{- 18} {s}^{2}$$

For a relative dielectric constant of 3, this becomes

$$\frac{Dwd}{t} = 4.7 \times {10}^{- 2} {m}^{2}$$

The plot below shows the overlap width for resonance at 64 MHz as a function of dielectric thickness for a relative dielectric constant of 3. The results for three ring diameters are shown. [image]
We can see that there are quantitative differences between the ring and sheet results, but they are in good qualitative agreement.

To test the actual performance of a spiral coating as shown in FIG. 2, several depositions were made. In these experiments a coating apparatus as shown in FIG. 3 was set up using two sputtering sources, each with a diameter of 5 cm. Source 52 was aluminum oxide and source 54 was silver and they were each placed approximately 4 cm from the substrate 56. The barrier 58 was made from stainless steel and it had a mask 60 affixed to it on the silver coating side that restricted the width of the silver coating, d, to 5.6×10⁻³ m. The aluminum oxide was sputtered at a power of 200 W and the silver was sputtered at a power of 80 W. The sputtering gas was Ar and the pressure was 3.5 mTorr. The deposition rate of both materials and the relative dielectric constant of the aluminum oxide were measured. At a rotation speed of 0.5 revolutions per hour the aluminum oxide thickness t was approximately 4.4×10⁻⁷ m and the silver thickness was approximately 8×10⁻⁶ m. The value of εᵣ for the aluminum oxide was determined to be 10.3 by using a C-V measurement technique well known to those skilled in the art. The substrate was a glass tube with a diameter D of 7×10⁻³ m.

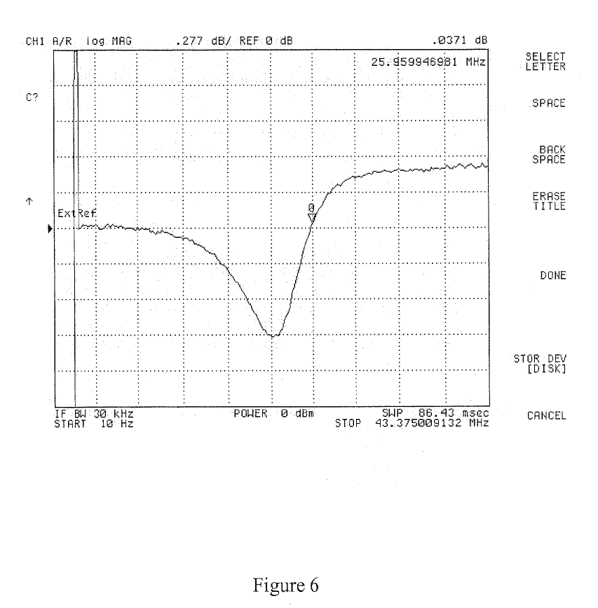

The two materials were simultaneously deposited while the substrate was rotated through 290 degrees and the resulting value for the overlap width w was approximately 1.8 mm. The resonant frequency of the coating was measured using an impedance analyzer in a method well known to those skilled in the art and the resulting pick-up coil signal amplitude as a function of frequency is shown in FIG. 6. The measured resonance occurred at 26 MHz and the resonance frequency predicted on the basis of Equation 1 is 37 MHz. The difference may be due to errors in our estimate of the dielectric thickness or overlap area. Nevertheless, the distinct resonance in FIG. 6 shows that the inventive method results in a coating structure having the properties of an RLC circuit.

In order to allow the coatings to accommodate the large strains in use mentioned earlier, it may be advantageous to use polymeric insulating layers, which are generally more flexible than inorganic insulating materials. Moreover, some of these materials, such as parylene, are already used to coat medical devices for other purposes. And in order to allow the conducting layer to accommodate the strains, it may be possible to deposit Nitinol with the same properties as the underlying device. Alternatively, as described in published US Patent Applications 20060015026 and 20060004466, both of which are incorporated herein by reference, for any and all purposes, we have found that by depositing porous layers of electrically conducting materials the resulting coatings can undergo the large strains produced in the use of such implantable devices without delamination.

The process disclosed above has been described in one preferred embodiment in which the process is used to produce coatings on devices that may be implanted in biological organisms. However it will be apparent to those skilled in the art that the process can also be used to coat other objects such as discrete electronic circuit components, objects requiring shielding from electromagnetic radiation, antennas for radiating and so on.

As previously discussed, the coated ring assembly embodiments disclosed above in FIG. 2 and FIG. 2A are in terms of simple single coated conducting rings so as to simplify the drawings for the detailed description of the coated layer embodiments therein. Referring again to FIGS. 1A, 1B, and 1C, any of the coating embodiments depicted in FIG. 2 or 2A may be coated on one or more of the sawtoothed shaped circumferential loops 110 of stents 100 and 150. Any of the coated layer embodiments may also be applied to any of the closed loop conducting paths of stents 100 and 150 as has been discussed elsewhere in this specification.

Additionally, it should be obvious to those skilled in the art that the coated layer embodiments described in terms of coatings disposed on a single conducting circular ring may also be extended to any device comprised of a conducting substrate with one or more holes therein, wherein electromagnetic radiation is incident on the device.

Some of the conducting materials that may be used for the top-most conducting layers in all of the coated layer embodiments disclosed above in this specification may be incompatible with the biological tissues in which the coated devices are implanted. If the top-most conducting layer is incompatible with the biological tissue in which the coated device is implanted, the device will be coated with a final insulating layer, which isolates the top-most conducting layer from the biological tissue in which the device is implanted. Such a final coated layer is not shown in any of the figures of embodiments as described above, but it should be understood that those embodiments will additionally comprise such a final coated layer when required for compatibility of the implanted device with the surrounding biological tissue. Such a final insulating coated layer will not affect the advantageous affect of the underlying coated layers.

Although the present invention has been described in considerable detail with reference to certain preferred versions thereof, other versions are possible. For example, multiple sources of electrically conducting material and/or multiple sources of electrically insulating materials may be a part of a system according to the invention. Therefore, the spirit and scope of the appended claims should not be limited to the description of the preferred versions contained herein.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

What is claimed is:

1. A system for coating a medical device for use within a subject so that the device is capable of being imaged using magnetic resonance, the system comprising:
a medical device;
a source of an electrically conducting material positioned to coat at least a portion of the medical device;
a source of an electrically insulating material positioned to coat at least a portion of the medical device;
at least one shield isolating the electrically conducting material from the electrically insulating material; and
a device for rotating the medical device relative to the conducting material and the insulating material.

2. The system of claim 1 wherein said source of an electrically insulating material is a physical vapor deposition source.

3. The system of claim 1 wherein said electrically insulating material is a curable monomer.

4. The system of claim 1 wherein said electrically insulating material is an evaporated polymer.

5. The system of claim 1 wherein said source of an electrically insulating material is a plasma polymerization source.

6. The system of claim 1 wherein said source of an electrically conducting material is a physical vapor deposition source.

7. The system of claim 1 wherein the electrically conducting material comprises at least one of Au, Ag, Cu, Ti, Ni, Pt or Pd.

8. The system of claim 1 in wherein the electrically conducting material comprises a shape memory alloy.

9. The system of claim 8 wherein the alloy is Nitinol.

10. The system of claim 1 wherein the electrically insulating material comprises at least one of a metal oxide or a nitride.

11. The system of claim 10 wherein the metal oxide or the nitride comprises one of Al₂O₃, AlN, TiO₂ or Ta₂O₅.

12. The system of claim 1 wherein the electrically insulating material is a polymer.

13. The system of claim 1 wherein the electrically insulating material is plasma polymerizable.

14. A method for coating a medical device for use within a subject so that the device is capable of being imaged using magnetic resonance, the method comprising:
positioning a source of an electrically conducting material to coat at least a portion of a medical device;
positioning a source of an electrically insulating material to coat at least a portion of the medical device;
shielding the electrically conducting material from the electrically insulating material; and
rotating the medical device relative to the electrically conducting material and the electrically insulating material.

15. The method of claim 14 wherein said source of an electrically insulating material is a physical vapor deposition source.

16. The method of claim 14 wherein said electrically insulating material is a curable monomer.

17. The method of claim 14 wherein said electrically insulating material is an evaporated polymer.

18. The method of claim 14 wherein said source of an electrically insulating material is a plasma polymerization source.

19. The method of claim 14 wherein said source of an electrically conducting material is a physical vapor deposition source.

20. The method of claim 14 wherein the electrically conducting material comprises at least one of Au, Ag, Cu, Ti, Ni, Pt or Pd.

21. The method of claim 14 wherein the electrically conducting material comprises a shape memory alloy.

22. The method of claim 21 wherein the alloy is Nitinol.

23. The method of claim 14 wherein the electrically insulating material comprises at least one of a metal oxide or a nitride.

24. The method of claim 23 wherein the metal oxide or the nitride comprises one of Al₂O₃, AlN, TiO₂ or Ta₂O₅.

25. The method of claim 14 wherein the electrically insulating material is a polymer.

26. The method of claim 14 wherein the electrically insulating material is plasma polymerizable.

27. The method of claim 14 further compromising coating the medical devices with a porous electrically conducting material.

28. The method of claim 14 further comprising:
coating the medical device with an electrically insulating material; and
coating the medical device with an electrically conducting material.

29. The method of claim 28 wherein the coatings on the medical device resonate at the applied frequency of a magnetic resonance imaging device.
